(19) United States
(12) Patent Application Publication
(10) Pub. No.: US 2011/0082549 A1
(43) Pub. Date: Apr. 7, 2011

(54) MINERALIZED THREE-DIMENSIONAL BONE CONSTRUCTS FORMED FROM OSTEOBLASTS

(75) Inventors: Mark S.F. Clarke, Houston, TX (US); Mark R. Brinker, Houston, TX (US); Alamelu Sundaresan, Sugarland, TX (US)

(73) Assignee: OSTEOSPHERE, LLC, Houston, TX (US)

(21) Appl. No.: 12/865,568

(22) PCT Filed: Feb. 2, 2009
(86) PCT No.: PCT/US2009/032843

§ 371 Date: Dec. 15, 2010

Related U.S. Application Data
(60) Provisional application No. 61/025,591, filed on Feb. 1, 2008.

(51) Int. Cl. A61F 2/28; C12Q 1/02; C12N 5/071

(52) U.S. Cl. 623/16.11; 435/29; 435/394

(57) ABSTRACT

The present disclosure provides ex vivo-derived mineralized three-dimensional bone constructs. The bone constructs are obtained by culturing osteoblasts under randomized gravity vector conditions. Preferably, the randomized gravity vector conditions are obtained using a low shear stress rotating bioreactor, such as a High Aspect Ratio Vessel (HARV) culture system. The bone constructs of the disclosure have utility in physiological studies of bone formation and bone function, in drug discovery, and in orthopedics.

### CLAIM OF PRIORITY

The present application for patent claims priority to U.S. Provisional Application No. 61/025,591 entitled “Mineralized Three-Dimensional Bone Constructs Formed From Osteoblasts” filed Feb. 1, 2008, assigned to the assignee hereof, and hereby expressly incorporated by reference herein.

### FIELD OF THE INVENTION

The invention relates to ex vivo-derived mineralized three-dimensional bone constructs which replicate natural bone. The bone constructs of the disclosure are formed by culturing osteoblasts.

### BACKGROUND

One of the central problems associated with studying both the normal and pathophysiology of bone is that as an organ system it is slow growing and the time to show an observable response to a particular stimulus is relatively long. The nature of the mineralized tissue matrix of bone in vivo and its complex architecture also presents several technical problems associated with how experimental observations can be made. At present, truly informative studies designed to understand bone physiology have relied primarily on the removal of samples of bone tissue from normal or diseased tissue either in a clinical setting or from experimental animal models.

In U.S. Provisional Patent Application Ser. Nos. 60/988,000, 60/988,008, 60/988,017, 60/988,020, 60/988,025, and 60/787,431, and in U.S. patent application Ser. No. 11/693,662, and in PCT Application Serial No. PCT/US2007/65542, each of which is incorporated herein by reference in its entirety, ex vivo-derived mineralized three-dimensional bone constructs are disclosed, along with methods for their formation and methods for their use. The bone constructs of these prior disclosures are formed from osteoclasts and osteoblasts. The inner core of bone constructs formed from these cell types comprises a three-dimensional crystalline matrix that stains positively with Alizarin Red S stain and with the von Kossa histochemical stain, indicating that it comprises mineral elements observed in normal human bone in vivo, including calcium, phosphates, and carbonates. The inner core also comprises osteoblasts and/or osteocytes embedded within the crystalline matrix. Osteocytes are developmentally inactive cells found only in native bone tissue in vivo and are believed to be formed from osteoblasts that have become trapped in the crystalline matrix. The inner core is “bone like” in appearance by visual inspection, in certain important respects resembling trabecular bone (also known in the art as “spongy bone”). The inner core, however, lacks osteoclast cells which are instead found, along with osteoblasts, in an outer surface layer that surrounds the inner core.

The present inventors have now realized that since the inner core of the aforementioned bone constructs lack osteoclasts, it will be possible to form bone constructs using osteoblasts without also using osteoclasts.

### SUMMARY

In one aspect, the disclosure provides ex vivo-derived mineralized three-dimensional bone constructs, each comprising a spheroid of between about 100 μm and about 4 mm in diameter. The spheroids comprise an inner core surrounded by an outer layer. The inner core of the spheroid is comprised of osteoblasts, osteocytes, or both osteoblasts and osteocytes embedded within a crystalline matrix. The crystalline matrix is comprised of calcium, phosphates, and carbonates. The outer layer comprises osteoblasts and lacks osteoclasts.

### DETAILED DESCRIPTION

In one aspect, the present disclosure provides mineralized three-dimensional bone constructs (sometimes referred to as “OsteoSpheres” or “bone constructs”). The mineralized three dimensional constructs of the disclosure are “bone like” in appearance by visual inspection, in certain important respects resembling trabecular bone (also known in the art as “spongy bone”). In preferred embodiments, the mineralized three-dimensional bone constructs of the disclosure are macroscopic in size and are approximately spheroidal in shape, preferably between about 100 μm and about 4 mm in diameter; however, larger and smaller bone constructs are specifically contemplated. The bone constructs of the disclosure are produced from osteoblast cells without the participation of osteoclast cells.

The bone constructs of the disclosure comprise an inner core surrounded by an outer layer. The inner core of the bone constructs of the disclosure comprises a three-dimensional crystalline matrix that stains positively with Alizarin Red S stain and with the von Kossa histochemical stain, indicating that it comprises mineral elements observed in normal human bone in vivo, including calcium, phosphates, and carbonates. The inner core also comprises osteoblasts and/or osteocytes embedded within the crystalline matrix, and is preferably devoid of necrotic tissue. Osteocytes are developmentally inactive cells found only in native bone tissue in vivo and are believed to be formed from osteoblasts that have become trapped in the crystalline matrix. The osteoblasts in the bone constructs of the disclosure can be obtained from any mammalian species, but are preferably obtained from humans.

The outer layer of the bone constructs of the disclosure comprises osteoblasts and does not include osteoclasts.

In another aspect, the disclosure provides methods for producing the mineralized three-dimensional bone constructs. In general, the bone constructs of the disclosure are produced by culturing osteoblasts under randomized gravity vector conditions (approaching those conditions that cultured cells experience during microgravity culture) in a matrix-free culture medium. Osteoblasts, preferably primary human osteoblasts, may also be obtained by techniques well known in the art, and may also be obtained from commercial sources (for example, from PromoCell, Inc. and from Cambrex/Lonza, Inc.). A “matrix-free culture medium” is a cell culture medium which does not include carrier material (such as microcarrier beads or collagen gels) onto which osteoblasts can attach. Suitable cell culture media include Eagle's Minimal Essential Medium (EMEM) or Dulbecco's Modified Eagle's Medium (DMEM), preferably supplemented with fetal bovine serum (FBS). Preferably, the matrix-free culture medium also comprises osteoblast growth supplements such as ascorbic acid. Example 2 includes a description of one suitable matrix-free culture medium.

The osteoblasts are cultured under randomized gravity vector conditions effective to achieve the formation of osteoblast aggregates. The osteoblast aggregates are then further cultured under randomized gravity vector conditions to increase the osteoblast aggregates size.

After a predetermined time, the osteoblast aggregates are cultured under randomized gravity vector conditions in a matrix-free mineralization culture medium. A “matrix-free mineralization culture medium” is a cell culture medium that includes one or more mineralization agents, such as osteoblast differentiation factors, that induce osteoblasts to produce crystalline deposits (comprising calcium, phosphate, and carbonates) but which does not include carrier material (such as microcarrier beads and collagen gels) onto which osteoblasts can attach. For example, in one embodiment, a matrix-free mineralization culture medium comprises FBS-supplemented EMEM or DMEM, supplemented with the osteoblast differentiation factors. Osteoblast differentiation factors include beta-glycerophosphate and hydrocortisone-21-hemisuccinate. Preferably, the matrix-free mineralization culture medium also includes osteoblast growth supplements such as ascorbic acid. For example, in one embodiment the matrix-free mineralization culture medium comprises FBS-supplemented DMEM, beta-glycerophosphate, ascorbic acid, and hydrocortisone-21-hemisuccinate. Example 2 includes a description of one suitable matrix-free mineralization medium.

In preferred embodiments, randomized gravity vector conditions are obtained by culturing osteoblasts in a low shear stress rotating bioreactor. Such bioreactors were initially designed to mimic some of the physical conditions experienced by cells cultured in true microgravity during space flight. In general, a low shear stress rotating bioreactor comprises a cylindrical culture vessel. One or more ports are operatively associated with the lumen of the vessel for the introduction and removal of cells and culture media. The cylindrical culture vessel is completely filled with a culture medium to eliminate head space. The cylindrical culture vessel rotates about a substantially central horizontal axis. The resulting substantially horizontal rotation occurs at a rate chosen so that (1) there is essentially no relative motion between the walls of the vessel and the culture medium; and (2) cells remain in suspension within a determined spatial region of the vessel such that they experience a continuous “free fall” through the culture medium at terminal velocity with low shear stress and low turbulence. This free fall state may be maintained continuously for up to several months in some applications described in the prior art. The continuous orbital movement of the medium relative to the cells also allows for highly efficient transfer of gases and nutrients.

In some embodiments, the diameter of the cylindrical culture vessel is substantially greater than its height. Such cylindrical culture vessels are often referred to in the art as High Aspect Ratio Vessels (HARVs). For example, a HARV having a volume of 10 mL may have a diameter of about 10 cm and a height of about 1 cm. At least a portion of the vessel walls may be comprised of a gas permeable membrane to allow gas exchange between the culture medium and the surrounding incubator environment. A suitable HARV is described in, for example, U.S. Pat. No. 5,437,998, incorporated by reference herein in its entirety. One commercial embodiment of a HARV is the Rotating Cell Culture System (RCCS) available from Synthecon, Inc.

In some embodiments, the diameter of the cylindrical culture vessel is substantially smaller than its height. Such cylindrical culture vessels are often referred to in the art as Slow Turning Lateral Vessels (STLVs). STLVs typically have a core, comprised of a gas permeable membrane, running through the center of the cylinder in order to allow gas exchange between the culture medium and the surrounding incubator environment. STLVs are available from Synthecon, Inc.

The use of low shear stressing rotating bioreactor culture systems is described in, for example, Nickerson et al., Immunity. 69:7106-7120 (2001); Carterson et al., Infection & Immunity. 73(2):1129-40 (2005); and in Goodwin et al. U.S. Pat. No. 5,496,722, each of which is specifically incorporated herein by reference in its entirety.

In one embodiment, osteoblasts are introduced into a cylindrical culture vessel in matrix-free culture medium. The absolute number of cells introduced into the cylindrical culture vessel may be varied. For example, in some embodiments, about 2 million osteoblasts are introduced; in other embodiments about 4 million osteoblasts are introduced; and in still further embodiments about 8 million osteoblasts are introduced. The absolute number of cells can be varied in order to vary the size and the number of osteoblast aggregates formed. In addition, other cell types (other than osteoclasts) may also be introduced into the cylindrical culture vessel. For example, bone marrow stroma and stem cells may be cultured along with the osteoblasts.

One or more cell types may optionally be labeled with a cell-tracking marker, such as a fluorescent cell-tracking dye, prior to their introduction into the cylindrical culture vessel. In this way, it is possible to determine the location of the individual cell types during, or at the conclusion of, the formation of the bone constructs. For example, fluorescent CellTracker dyes, available from Invitrogen, Inc., may be used in conjunction with fluorescence microscopy techniques, such as confocal fluorescence microscopy. If more than one cell type is labeled, then they are labeled with different colored dyes so that each cell type can be tracked independently.

Cells are then cultured in the matrix-free culture medium in the cylindrical culture vessel during substantially horizontal rotation to form osteoblast aggregates. The rate of substantially horizontal rotation during the aggregation phase is chosen so that both (1) low shear conditions are obtained; and (2) the osteoblasts are able to coalesce and form osteoblast aggregates. The rate of substantially horizontal rotation may be selected by monitoring the cylindrical culture vessel and by monitoring the cells and osteoblast aggregates in the cylindrical culture vessel (for example using microscopy), to insure that the cells and osteoblast aggregates are not sedimenting (which may be caused by too low a rate of rotation) or experiencing mechanical or excessive hydrodynamic shear stress. In embodiments in which a HARV is used, osteoblasts may form a “boundary” layer situated in the middle of the HARV during the aggregation phase.

Preferably, the rate of substantially horizontal rotation during the aggregation phase is lower than the rate typically used for culturing cells. For example, in embodiments where the cylindrical culture vessel is a 10 mL HARV having a diameter of about 10 cm and a height of about 1 cm, substantially horizontal rotation at less than about 14 revolutions per minute (rpm) may be used. More preferably, substantially horizontal rotation at less than about 12 rpm is used. In certain preferred embodiments, substantially horizontal rotation at between about 1 rpm and about 4 rpm is used. In one specific embodiment, substantially horizontal rotation at about 2 rpm is used. Note that the aforementioned rpm values are provided with reference to a 10 mL HARV having the aforementioned dimensions. The rpm values will vary depending on the volume and dimensions of the cylindrical culture vessel. The rpm values during the aggregation phase for all such vessels are easily determined using the aforementioned methodology.

Without being bound by a particular theory or mechanism, it is believed that the use of a matrix-free culture medium allows the use of rates of rotation that are substantially lower than previously reported in the art for culturing mammalian cells in a low shear stress rotating bioreactor. The use of low rotation rates, in turn, is believed for the first time to promote efficient association of osteoblasts into osteoblast aggregates, and to promote three-dimensional organization of this cell type within the aggregate. Thus, the organization of the osteoblast aggregate is not constrained or influenced by an exogenous carrier material, but rather by native cell-cell interaction. Consequently, the three-dimensional organization of the osteoblasts is physiologically realistic.

The rate of substantially horizontal rotation may optionally be adjusted periodically during the aggregation phase in order to compensate for the increase in the sedimentation velocity (which is a function of volume and density) of the forming osteoblast aggregates, thereby maintaining the osteoblast aggregates in low shear “free fall” and preventing impact with the vessel wall.

The osteoblast aggregation phase proceeds for a period of time sufficient to produce the desired size of osteoblast aggregates. Aggregate formation may be monitored during the aggregation phase by visual inspection, including through the use of microscopy. It will be apparent from the disclosure that the size of the osteoblast aggregates is also dependent on the number of cells that are initially introduced into the cylindrical culture vessel, the length of time allowed for aggregation, as well as the rotation rate. In one example, the aggregation phase is allowed to proceed for between about 24 hours and about 48 hours.

Once osteoblast aggregates of the desired size have formed, the osteoblast aggregates are preferably further cultured in the cylindrical culture vessel during substantially horizontal rotation for a period of time sufficient to allow the osteoblast aggregates to grow to a desired size through cell proliferation. For example, the further culturing of the osteoblast aggregates may proceed for between about 5 and about 7 days and may lead to grown osteoblast aggregates having a diameter from between about 100 μm and about 4 mm. The resultant osteoblast aggregates are sometimes referred to herein as “spheroids.” Preferably, the rate of substantially horizontal rotation during the further culturing is higher than the rate during the aggregation phase, but still provides low shear conditions in the cylindrical culture vessel. For example, a rotation rate of between about 9 rpm and about 16 rpm, preferably about 14 rpm, may be used during further culturing for the 10 mL HARV exemplified above. The rate of substantially horizontal rotation may optionally be adjusted periodically during the further culturing phase in order to compensate for the increase in the sedimentation pathway of the osteoblast aggregates as they grow in size (and hence undergo changes in volume and density), thereby maintaining the growing osteoblast aggregates in low shear “free fall” and preventing impact with the vessel wall.

Once osteoblast aggregates have attained a desired size, a matrix-free mineralization culture medium is introduced into the cylindrical culture vessel and the osteoblast aggregates are cultured during substantially horizontal rotation until they become mineralized (either partially mineralized or fully mineralized), thereby forming the mineralized three-dimensional bone constructs of the disclosure. For example, the mineralization process may proceed for between about 7 days and about 21 days depending on the size of the osteoblast aggregates and the degree of mineralization required. Preferably, the rate of substantially horizontal rotation during such the mineralization process is higher than the rate during the aggregation phase, but still provides low shear conditions in the cylindrical culture vessel. For example, a rotation rate of between about 9 rpm and about 20 rpm, preferably about 14 rpm, may be used during the mineralization phase for the 10 mL HARV exemplified above. The rate of substantially horizontal rotation may optionally be adjusted periodically during the mineralization phase in order to compensate for the increase in the sedimentation pathway of the osteoblast aggregates as they increase in mass, thereby maintaining the mineralizing osteoblast aggregates in low shear “free fall” and preventing impact with the vessel walls.

Mineralized three-dimensional bone constructs are harvested once they have achieved the desired size and mass. In cylindrical culture vessels with one or more access ports, the bone constructs are removed through a part. When the bone constructs exceed the diameter of the port, the vessel is disassembled to remove the bone constructs.

The mineralized three-dimensional bone constructs of the disclosure mimic trabecular bone in many important aspects. The bone constructs of the disclosure therefore have a great many uses in the fields of, for example, physiology research and development, pharmaceutical research, and orthopedics. Without limitation, these include the direct benefit of developing a model for studying both normal bone physiology and the pathological responses observed in disease states such as osteoporosis, as well as providing a highly economical platform for drug development as it relates to the treatment of bone diseases.

The bone constructs of the disclosure also can be used for autologous grafts. Specifically, diseased or missing bone may be replaced with ex-vivo-derived mineralized three-dimensional bone constructs in which the component osteoblasts are harvested from healthy bone and peripheral blood lymphocytes of the patient requiring the bone graft. Examples of pathologies where the bone constructs of the disclosure have therapeutic utility include fractures, non-unions of fractures, congenital deformities of bone, bone infections, bone loss, segmental bone defects, bone tumors, metabolic and endocrine disorders affecting bone, and tooth loss.

The bone constructs of the disclosure can also be used for allogenic (allograft) grafts. Specifically, diseased or missing bone can be replaced with ex vivo-derived mineralized three-dimensional bone constructs in which the component osteoblasts are harvested from healthy bone and peripheral blood lymphocytes of another donor for the benefit of a patient requiring bone graft. Examples of pathologies where the bone constructs of the disclosure have therapeutic utility include fractures, non-unions of fractures, congenital deformities of bone, bone infections, bone loss, segmental bone defects, bone tumors, metabolic and endocrine disorders affecting bone, and tooth loss.

Because the bone constructs of the disclosure closely resemble bone formed in vivo, it is expected that they produce unique factors and/or cytokines essential for bone remodeling. Accordingly, the bone constructs of the disclosure serve as a source for identification and harvesting of these factors.

The bone constructs of the disclosure may also be used to study the interface between prosthetic devices/materials and bone tissue.

Sensors or stimulation devices may be incorporated into the bone constructs of the disclosure, and the resulting constructs implanted into bone tissue in vivo.

The bone constructs of the disclosure also may be used in the production of large structures of specific dimensions for “form-fitted” applications such as replacement of large regions of the skeleton. This may be achieved using a combination of tissue scaffolding/synthetic support materials embedded with numerous bone constructs to generate a much larger composite tissue aggregate.

The bone constructs of the disclosure also provide a low cost alternative in which to study the effects of microgravity, and of other space environment insults, such as radiation, on the process of bone formation/bone loss.

The following examples are not to be construed as limiting the scope of the invention disclosed herein in any way.

### EXAMPLES

### Example 1

### A Method for Producing Bone Constructs from Osteoblasts without the Participation of Osteoclasts

Osteoblast are first isolated from a healthy patient and then inoculated into a modified High Aspect Rotating Vessel (HARV) with a matrix-free culture medium. Cells are allowed to aggregate at a rotation speed (typically 2 rpm) much lower than that commonly used for the culture of mammalian cells. Low speed promotes aggregation of the cells in the early stages of aggregate formation. After the aggregation period is over, the rotation speed of the High Aspect Rotating Vessel is increased. This allows the bone construct to grow into spheroids in a state of “free fall”. The mineralization step (160) is then initiated by exchanging the initial matrix-free culture medium for a matrix-free mineralization culture medium, which initiates the production of a calcified crystalline matrix in the center of the tissue aggregate. The bone constructs are then characterized. The spatial arrangement of the different cell types is observed by confocal microscopy imaging. The cells are visualized with Z-series confocal imaging by pre-labeling the initial cell constituents of the construct with green fluorescent cell tracker probe. The presence of calcium, phosphate and carbonate is revealed by using Alizarin red S stain and Kossa histochemical stain, while the presence of nucleated cells embedded in the crystalline matrix is revealed by nuclear staining Immuno-staining of the construct for markers such osteocalcein (an osteoblast marker) and alkaline phosphate is also performed.

### Example 2

### Production of Bone Constructs in a HARV

Cryopreserved primary normal human osteoblast cells may be purchased from the Cambrex Corporation (East Rutherford, N.J.) and are stored frozen under liquid nitrogen until needed.

Osteoblast cells are rapidly thawed by placing the vial in a 37° C. oven, removing the cell suspension from the vial and placing it in a 15 ml centrifugation tube and then diluting the cell suspension with 10 ml of Dulbecco's Modified Essential Medium (DMEM) supplemented with 10% (v/v) fetal bovine serum (10% FBS-DMEM). The cells are then collected by centrifugation at 100×g for 5 min at 4° C. The supernatant is then removed and the cell pellet is resuspended by gentle tituration in 10 ml of fresh 10% FBS-DMEM supplemented with 5 μM ascorbic acid and 1 mg/ml GA-1000 (gentamicin/amphotericin B mixture). This process is carried out to wash away the cryopreservatives in which the osteoblast cells are been frozen.

The resulting cell suspension is then inoculated into a T-75 tissue culture flask and incubated at 37° C. in a 5% CO₂ atmosphere tissue culture incubator for a total period of seven days, with the medium being exchanged every three days. After seven days the osteoblast culture approaches confluence and the osteoblast cells are harvested by removing the cells from the surface of the flask using trypsin/EDTA digestion followed by collection of the cells by centrifugation as above. The cell pellet is then gently resuspended in 20 ml of fresh 10% FBS-DMEM supplemented with 5 μM ascorbic acid and 1 mg/ml GA-1000. The resulting cell suspension is then inoculated into two T-75 tissue culture flasks and again cultured for an additional seven days. This process of osteoblast cell expansion is continued until the cells have reached passage 5 (i.e. five expansion/population doubling cycles).

When the osteoblast cells have reached Passage 5 in culture they are harvested using trypsin/EDTA digestion followed by collection of the cells by centrifugation as above. The cell pellet is then gently resuspended in 10 ml of fresh 10% FBS-DMEM supplemented with 5 μM ascorbic acid, 100 U/ml penicillin and 100 ug/ml streptomycin, penicillin/streptomycin being substituted for GA-1000 at this point due to the potential negative effects of gentamicin on the capability of osteoblast cells to produce mineralized extracellular matrix. The resulting osteoblast cell suspension is counted using a hemacytometer to ascertain the number of osteoblast cells/ml. An aliquot of cell suspension containing a total of six million osteoblast cells is removed and placed in a separate 15 ml centrifugation tube.

The volume of medium in the centrifuge tube is then adjusted to a total of 10 ml by the addition of fresh 10% FBS-DMEM supplemented with 5 μM ascorbic acid, 100 U/ml penicillin and 100 ug/ml streptomycin.

The resulting cell suspension is then inoculated into a 10 ml rotating cell culture system (RCCS) flask (also know as a High Aspect Ratio Vessel—HARV) (Synthecon, Inc.) and horizontally rotated at 2 RPM for a period of 24 hr to allow coalescence of the osteoblast cells into a solid, three dimensional tissue construct (osteoblast aggregates). After a period of 24 hr, the rotation speed of the HARV is increased to 14 RPM in order ensure that the tissue construct is maintained in an optimal position within the HARV, namely not touching or hitting the sides of the rotating HARV rather in a state of “free-fall” within the medium contained within the rotating HARV. The cell medium within the HARV is exchanged with 10 ml of fresh 10% FBS-DMEM supplemented with 5 μM ascorbic acid, 100 U/ml penicillin, and 100 ug/ml streptomycin (a matrix-free culture medium) after every fourth day of culture.

After a period of seven days of culture in the HARV under the above conditions the medium is exchanged for 10 ml of fresh 10% FBS-DMEM supplemented with 5 μM ascorbic acid, 100 U/ml penicillin, 100 ug/ml streptomycin, 200 μM hydrocortisone-21-hemisuccinate and 10 mM beta-glycerophosphate (a matrix-free mineralization culture medium). The hydrocortisone-21-hemisuccinate and beta-glycerophosphate is added to the medium to induce mineralization of the tissue construct by the osteoblasts. The cell medium within the HARV is exchanged with 10 ml of fresh 10% FBS-DMEM supplemented with 5 μM ascorbic acid, 100 U/ml penicillin, 100 ug/ml streptomycin, 200 μM hydrocortisone-21-hemisuccinate and 10 mM beta-glycerophosphate every fourth day until the tissue construct is harvested.

### Example 3

### Ex-Vivo Production of Human Demineralized Bone Matrix

Bone grafting is a surgical procedure used to treat a variety of long bone, spine-related and periodontal problems. The procedure involves reconstruction/fusion of bones or spinal vertebrae, or grafting of bone voids or defects, the ultimate goal being mechanical stability of the repaired bone. In the long bones, a bony defect may exist following trauma, tumor, infection, or other causes. In the spine, various pathologies may require bone grafting such as following trauma to the vertebrae, protrusion/degeneration of the intervertebral disc, abnormal curvatures of the spine (i.e. scoliosis or kyphosis) or a weak or unstable spine caused by infections or tumors. Another area in which bone grafting has become a viable clinical approach is periodontal reconstruction of the maxilla or mandible bones prior to dental implantation.

The standard means of bone grafting is to insert actual bone or bone substitute material within a bony defect, between the vertebrae or within the maxilla or mandible bones in order to fill a void or promote fusion of the existing bones. The procedures are commonly performed in conjunction with mechanical fixation using specialized hardware such as plates, screws, rods, etc. The purpose of the hardware is to immobilize the long bones or vertebrae until the healing/fusion process has occurred. It is important to note that the bones or vertebrae being fused are already compromised in terms of their structural integrity and are usually undergoing matrix degeneration and bone resorption, without the concomitant bone formation process that occurs during normal bone remodeling in healthy individuals. In the case of periodontal reconstruction of the jaw prior to dental fixture implantation, this procedure usually involves reconstruction of the maxilla or mandible bones at a site where the existing bone matrix is compromised and unable to mechanically support the implantation of a screw or post that in turn is used to attach a dental fixture.

There are several major types of bone graft material used in long bone and spinal fusion procedures. The most clinically effective bone graft material is autogenous bone, usually harvested from the patient's iliac crest, which is then implanted within a long bone defect or between vertebrae to be fused. However, this approach involves a second painful surgical procedure to harvest the autogenous bone graft material and increases the risk of patient complications unrelated to the primary procedure.

The use of demineralized bone matrix (DBM) as a substitute for autogenous bone graft or to augment or extend the amount of bone graft material available for a particular orthopedic or periodontal surgical procedure is becoming increasingly common. DBM is primarily osteoconductive while displaying some osteoinductive properties. DBM is obtained from both human and animal sources and is produced by the acid extraction of bone material resulting in the removal of most of the mineralized component while retaining the collagenous and non-collagenous proteins including a variety of growth factors. Such growth factors include the bone morphogenic proteins (BMP's) BMP-2 and BMP-7, both members of the TFG-beta super-family of growth factors. It has been suggested that the osteoinductive properties of DBM are directly related to their BMP content, the higher the amount of BMP's present, the greater the osteoinductive properties of the material, a concept known as proportional osteoinduction.

Usually, DBM is produced by physical disruption/grinding of bone material followed by acid extraction and washing resulting in a fine powder material that is then sterilized. This fine powder material is mixed immediately prior to use with a variety of agents to form a gel or paste for ease of handling during surgery and to aid in its application and retention within a bony defect. Such agents commonly include glycerol to make a paste or fibrin clot material to form a solid yet flexible/malleable material. However, the use of DBM in both orthopedic and periodontal procedures is limited by the variability in its osteoconductive and osteoinductive properties, with a wide variation in either or both of these parameters being observed depending on the original batch of material from which the DBM was originally produced.

From a theoretical perspective, optimal DBM would be human in origin, would contain large amounts of BMP's, more importantly contain a variety of BMP's at the concentration and relative ratios required to promote optimal formation of new bone tissue, have consistent and predictable osteoconductive and osteoinductive properties, while at the same time being in limitless supply. To date, DBM available for clinical use has failed to satisfy all of the above requirements primarily due to an inability to reliably produce DMB with such consistent and predictable properties from human sources due to a limited and varied raw material supply.

The mineralized three-dimensional bone constructs (sometimes referred to as “OsteoSpheres”) of the disclosure can be produced on demand in practically limitless supply from cryogenically stored human osteoblasts using the methods of the preceeding examples. As such, the raw material for the production of these OsteoSpheres can be carefully controlled for both quality and consistency.

The inventors have realized that during the process of OsteoSphere production in culture that the biochemical and cellular milieu generated within the Osteo Spheres during their formation, differentiation and mineralization ex vivo will follow the same cellular pathways that occur during normal bone formation in vivo. As such, OsteoSpheres formed from osteoblasts will display both the osteoconductive and osteoinductive properties of normal human bone. Based on the concept that both of these properties are generated as a function of the presence of BMP molecules within the mineral matrix, the inventors have also realized that the mineralized matrix of mature OsteoSpheres will not only contain a variety of BMP's, but will also contain the appropriate mixture and relative amounts of BMP's required to promote normal bone formation. It may also be expected that depending on the stage the OsteoSpheres are at in their culture cycle (i.e. immature non-mineralized OsteoSphere 0-7 days of culture; mature partially mineralized OsteoSpheres 7-14 days of culture; mature completely mineralized OsteoSpheres 14-21 of culture) the relative types and concentrations of BMP's being produced and sequestered in the OsteoSphere extra-cellular matrix components will reflect the optimal BMP mixture and concentration required for that phase of normal bone growth in vivo. For example, immature non-mineralized OsteoSpheres (0-7 days of culture) are analogous to bone material in vivo at the beginning of the bone formation process; mature partially mineralized OsteoSpheres (7-14 days of culture) are analogous to bone material in vivo at the beginning of the mineralization process but after the laying down of a collagenous matrix; mature completely mineralized OsteoSpheres (14-21 of culture) are analogous to mature bone. This progression is by definition controlled by cellular signals that include a variety of BMP's acting in concert to drive the process. Based on this information, it is entirely possible that production of DBM from OsteoSpheres with differing levels of maturation will have differing osteoinductive and osteoconductive properties. As such, DBM produced from OsteoSpheres of different maturation levels may be better suited to certain clinical applications than others based upon the particular clinical conditions. Specifically, a fresh fracture is likely to have a different optimum DBM profile than a long-standing nonunion or a bone void of the maxilla.

Described herein is a novel source and method for the production of human-derived DBM that has the advantage of being human in origin, contains physiologically active amounts of BMP's, contains a variety of physiologically active BMP's at the concentrations and relative ratios required to promote optimal formation of human bone tissue, that has consistent and predictable osteoconductive and osteoinductive properties and that can be produced in limitless quantities using a reproducible and controllable manufacturing process.

This process involves the production of OsteoSpheres (using, for example, the methods of the preceeding examples) of uniform diameter (from 200 micron up to 4 mm in diameter) that are grown for pre-determined lengths of time (i.e. 7, 14, or 21 days) under rotation in the presence of defined amounts of osteoblast differentiation (e.g. asorbic acid) and/or mineralization agents (e.g. beta-glycerolphosphate and hydrocortisone-21-hemisucinate) as disclosed in the preceeding examples. Osteospheres at various stages of maturation are removed from culture, washed in sterile phosphate buffered saline to remove any tissue culture medium components and physically disrupted using mechanical means (e.g. maceration and sonication) in a sterile acid solution to disrupt the embedded cells and remove acid soluble material. This acid washed material is then again washed in fresh sterile acid solution and dried by desiccation under sterile conditions. The resulting fine powder material (i.e. OsteoSphere-derived DBM) can then, if desired, be mixed with a sterile aqueous solution containing a mixture of ascorbic acid, beta-glycerolphosphate and hydrocortisone-21-hemisucinate to supplement the OsteoSphere-derived DBM with osteoblast differentiation and mineralization agents. The water is removed by desiccation and the product is packaged under sterile conditions.

### Example 4

### Development of a Human Colloidal Bone Graft Material

Bone grafting is a surgical procedure used to treat a variety of long bone and spine-related problems. The procedure involves fusion of bones or spinal vertebrae, the ultimate goal being mechanical stability. In the long bones, a bony defect may exist following trauma, tumor, infection, or other causes. In the spine, various pathologies may require bone grafting such as following trauma to the vertebrae, protrusion/degeneration of the intervertebral disc, abnormal curvatures of the spine (i.e. scoliosis or kyphosis) or a weak or unstable spine caused by infections or tumors.

The standard means of bone grafting is to insert actual bone or bone substitute material within a bony defect or between the vertebrae in order to promote fusion of the existing bones. This procedure is commonly performed in conjunction with mechanical fixation using specialized hardware such as plates, screws, rods, etc. The purpose of the hardware is to immobilize the long bones or vertebrae until the healing/fusion process has occurred. It is important to note that the long bones or vertebrae being fused are already compromised in terms of their structural integrity and are usually undergoing matrix degeneration and bone resorption.

There are several major types of bone material used in long bone and spinal fusion procedures. The most clinically effective bone graft material is autogenous bone, usually harvested from the patient's iliac crest, which is then implanted within a long bone defect or between vertebrae to be fused. Another material available is allograft (cadaveric) bone, which forms a meshwork into which the patient's own bone cells can migrate and ultimately form new bone. A more recent approach is the use of a collagen sponge impregnated with bone morphogenic protein-2 (BMP-2); this method is designed to promote new bone formation and also allows the patient's bone cells to infiltrate the sponge. The commercial name of this product is INFUSE™.

Of the above materials, autogenous bone graft is the most widely used and enjoys the highest clinical success rates. However, this approach involves a second painful surgical procedure to harvest the autogenous bone graft material and increases the risk of patient complications unrelated to the primary procedure. Allograft bone grafting does not require a second surgical procedure but has a lower rate of clinical success due to the requirement for the patients own bone to provide the cells required to infiltrate the allograft in order for the fusion process to progress. Considering the already compromised state of the bones to be fused, the rate at which this cell infiltration/remodeling occurs is variable and hence the efficiency and completeness of fusion that occurs is also variable. Similar issues surround the use of the INFUSE™.

The ideal bone graft material for use in long bone fractures/nonunions/defects and spinal fusion procedures is one that has all of the characteristics of autogenous bone graft without the need for a second surgical procedure to harvest the material. In addition, the material should contain all those elements necessary to provide the optimal cellular environment for new bone formation and the promotion of fusion with the existing matrix of the long bone or vertebrae being treated. By providing these elements, bony fusion will occur in a predictable and efficient manner reducing the time required for the patient to heal.

We here describe a substance known as Colloidal Bone Graft (CBG), development of which is based on the understanding of the process of human bone formation gained in producing large three dimensional human bone constructs ex vivo. This CBG material is designed for use in long bone and spinal fusion procedures and comprises numerous mineralized three-dimensional bone constructs (e.g. approximately 200 microns in diameter prepared according to the any of the preceeding examples, and sometimes referred to as “OsteoSpheres”) contained in a sterile liquid that comprises a polymerizable biocompatible matrix, such as gelatin, collagen or alginate, the osteoblast differentiation agent, ascorbic acid, and the osteoblast mineralization agents, beta-glycerolphosphate and hydrocortisone-21-hemisucinate. This material is prepared immediately prior to use by mixing cryogenically stored, colloidal OsteoSpheres with a sterile solution of, for example, aqueous sodium alginate containing ascorbic acid, beta-glycerolphosphate and hydrocortisone-21-hemisucinate. Immediately prior to implantation, the alginate is induced to gel/polymerize by the addition of a predetermined amount of calcium in the form of a sterile aqueous solution of calcium chloride. The now complete Colloidal Bone Graft material is then injected/dispensed into the fracture or nonunion site, into a bony defect, or between vertebrae being fused.

It is envisioned that this Colloidal Bone Graft material may be used in conjunction with internal or external skeletal or vertebral fixation or some form of internal structural support between the bones or vertebrae as the polymerized colloidal bone graft material has little or no structural strength before healing and/or mineralization has occurred. The presence of a three dimensional matrix containing colloidal OsteoSpheres (that are by definition producing a host of cellular-derived signals such as bone morphogenic proteins) and exogenous osteoblast differentiation and mineralization agents (previously shown to promote bone formation ex vivo) approximates the optimal material for long bone or spinal fusion, namely autogenous bone graft. The elements contained in the Colloidal Bone Graft material will encourage not only early bone cell infiltration from the patient's already remodeling long bones or vertebrae but will promote rapid mineralization of the Colloidal Bone Graft material once infiltrated by the patient's own osteoblasts. By promoting these events, rapid, efficient and predictable bone to bone healing can be achieved.

### Example 4

### Production of Bone Morphogenetic Proteins using a Novel Tissue Culture Platform

Bones are organs made up of bone tissue (osseous tissue) as well as marrow, blood vessels, epithelium and nerves. Bone tissue (the mineralized component) refers specifically to the mineral matrix that makes up the rigid portion of the bone and provides the mechanical stability of the organ as a whole. The process of bone tissue formation is one that involves a variety of physiological signals, including mechanical loading and a myriad of biochemical signaling molecules that act in concert upon the cells that are responsible for bone matrix production.

One of the major classes of signaling molecules involved in directing the formation of bone tissue are the bone morphogenic proteins (BMPs) who belong to the transforming growth factor super family of molecules. Although BMPs have previously been shown to regulate the growth and differentiation of various cell types, including chondrocytes where they stimulate the production of collagenous extra-cellular matrix, their osteoinductive properties have received wide attention as a possible means of stimulating new bone growth in the repair of a variety of clinically relevant bony defects.

BMPs induce early mesenchymal progenitor cells to enter the osteogenic differentiation pathway and stimulate the production of both collagen and alkaline phosphatase by osteoblasts during the formation of new bone. One clinical approach has been to harness the osteoinductive properties of BMPs by applying them to the site of a fracture or bony defect in order to stimulate new bone formation by the existing damaged bone. For example, BMP-7 (also known as OP-1) and BMP-2 have both received FDA approval for use in certain orthopedic procedures and the use of these compounds is being explored in the repair of a variety of other bone pathologies. Both these compounds have received significant success in enhancing bone repair and demonstrate the clinical efficacy of this approach. These data indicate that the use of BMPs to enhance bone formation may have utility in a variety of other clinical situations.

The therapeutic use of BMPs in the field of orthopedics has been negatively impacted by difficulties in obtaining or producing large quantities of these proteins in a biologically active form. BMPs have been produced either from endogenous or recombinant sources. Bone and other tissues, such as cartilage, contain very small concentrations of mature BMPs. Several methods exist that are capable of extracting biologically active BMPs from raw bone material. However, these protocols are prolonged, time consuming and produce very low yields of biologically active BMPs. For example, it has been reported that 15 kg of raw bone material will only generate approximately 0.5 g of partially purified BMPs (Urist et al. Meth Enz 1987; 146:294-312).

Commercially available BMP preparations, such as those that contain BMP-2 or BMP-7 are based upon mammalian protein expression systems. Both human BMP-2 and BMP-7 have been expressed in CHO Chinese hamster ovary (CHO) cells. However, this approach has a low productivity and overall yield. Due to these low yields, recombinant BMPs that can be utilized in clinical procedures are currently very expensive.

From a theoretical perspective, optimal BMP production for clinical applications would consist of a cell-based manufacturing process producing biologically active BMP molecules from a human source that could be easily harvested and purified in a continuous or batch-type culture process. It is clear that BMPs do not act independently of each other during the osteoinduction process, rather act in concert not only with other BMPs, but with additional growth factors not of the BMP family. As such, it is very probable that optimal BMP production will only occur when the cellular milieu surrounding the cultured cells is providing the appropriate biochemical signals. This cellular milieu is, by definition that which exists during normal bone remodeling or repair.

The inventors have realized that during the process of OsteoSphere production in culture that the biochemical and cellular milieu generated within the Osteo Spheres during their formation, differentiation and mineralization ex vivo will follow the same cellular pathways that occur during normal bone formation in vivo. As such, OsteoSpheres formed from osteoblasts will display both the osteoconductive and osteoinductive properties of normal human bone. Based on the concept that both of these properties are generated as a function of the presence of BMP molecules within the mineral matrix, the inventors have also realized that the mineralized matrix of mature OsteoSpheres will not only contain a variety of BMP's, but will also contain the appropriate mixture and relative amounts of BMP's required to promote normal bone formation. It may also be expected that depending on the stage the OsteoSpheres are at in their culture cycle (i.e. immature non-mineralized OsteoSphere 0-7 days of culture; mature partially mineralized OsteoSpheres 7-14 days of culture; mature completely mineralized OsteoSpheres 14-21 of culture) the relative types and concentrations of BMP's being produced and sequestered in the OsteoSphere extra-cellular matrix components will reflect the optimal BMP mixture and concentration required for that phase of normal bone growth in vivo.

For example, immature non-mineralized OsteoSpheres (0-7 days of culture) are analogous to bone material in vivo at the beginning of the bone formation process; mature partially mineralized OsteoSpheres (7-14 days of culture) are analogous to bone material in vivo at the beginning of the mineralization process but after the laying down of a collagenous matrix; mature completely mineralized OsteoSpheres (14-21 of culture) are analogous to mature bone. This progression is by definition controlled by cellular signals that include a variety of BMP's acting in concert to drive the process.

We here describe a novel source and method for the production of human-derived BMPs from Osteo Spheres that are being maintained at different levels of differentiation and mineralization. This process involves the harvesting of BMPs from the tissue culture medium in which the OsteoSpheres are growing. OsteoSpheres of uniform diameter (from 100 micron up to 4 mm in diameter) are grown for pre-determined lengths of time (i.e. 7, 14, or 21 days) under rotation in the presence of defined amounts of osteoblast differentiation (e.g. asorbic acid) and/or mineralization agents (e.g. beta-glycerolphosphate and hydrocortisone-21-hemisucinate) as described in the preceeding examples. Osteospheres are maintained at various stages of maturation within a rotating cell culture system by maintaining them in medium without mineralization agents, stimulating mineralization for only a short period of time (i.e. 7 days) and then removing the mineralization agents, or in the fully mineralized mature state. Conditioned medium containing BMPs are continuously removed from the culture vessel and replaced with the relevant type of culture medium to maintain the pre-determined Osteo Sphere maturation state. In addition, it is contemplated that Osteospheres could be removed from the HARV after formation and kept in an appropriate bioreactor or culture setting or media for an extended period of time as they continue to secrete BMPs. In addition, OsteoSphere cultures can also be stimulated with additional growth factors that promote cellular activity associated with the osteoinductive phenotype. These include the hormone, PTH and the growth factor, basic fibroblast growth factor (i.e. bFGF or FGF-2). Addition of these factors to the medium of Osteo Spheres will stimulate and increase osteoblast activity that will be paralleled by an increase in production and release of BMPs. As with the vast majority of proteins that are secreted and that can act as autocrine signaling molecules, BMPs that are produced and secreted by the cells of the Osteo Sphere can then be sequestrated in the surrounding extra-cellular matrix leading to a down-regulation of BMP production via a negative feedback loop. As BMPs are produced within the three dimensional mass of the OsteoSpheres, culture conditions that promote removal of the secreted BMPs from that three dimensional matrix or displacement of sequestered BMPs from the extra-cellular matrix will increase overall amounts of BMPs that can be harvested from the medium. Examples of such strategies include addition of low molecular weight heparin to the culture medium to disrupt the binding of BMPs to heparin sulphate moieties in the extra-cellular matrix of the OsteoSpheres, the use of higher rotational rates to promote medium flow and exchange within the three dimensional structure of the OsteoSphere or even removing mature Osteo Spheres from rotational culture and placing them in a static, continuous perfusion culture system in order to achieve high rates of tissue culture medium exchange with the three dimensional matrix of the Osteo Sphere.

An additional means of stimulating BMP production in OsteoSpheres is the use of mechanical stimulation. It is widely understood that increases in bone mineral density in vivo are directly linked to increased levels of cyclical strain placed on the bone. This is the basis for the use of high load resistive exercise protocols for increasing bone mineral density in subjects suffering from bone loss due to reduced musculoskeletal loading associated with prolonged bed-rest, osteoporosis, aging-induced osteopenia and space flight. Recently, the use of low frequency vibration (i.e. between 10 and 60 Hz) has been demonstrated to induce increases in bone mineral density in a variety of animals, including turkeys and sheep. Similar increases in bone mineral density have been observed in human subjects exposed to low frequency vibration. Increases in bone mineral density during bone remodeling are known to be associated with an increase in bone formation (i.e. increased osteoblast activity) and a decrease in bone resorption (i.e. decreased osteoclast activity), and as such mechanical stimulation by definition must enhance the production of the various signaling molecules responsible for this modified cellular activity. As OsteoSpheres are an ex vivo preparation of human bone, exposing OsteoSpheres to similar mechanical stimulation in culture will result in similar up-regulation of the signaling molecules, such as BMP's, responsible for increased bone mineral density in vivo. This can be achieved by exposure of the constructs to pulses of low frequency vibration using a sonicator, increased hydrodynamic shear by cyclically increasing and decreasing the rotational speed at which OsteoSpheres are cultured or compression loading by placing mature, mineralized OsteoSpheres in a non-rotating yet mechanically dynamic tissue culture environment such as that embodied in the commercially available Flexcell Tissue Culture System.

As clinically relevant BMPs are known to have heparin-binding properties, extraction and purification of biologically active BMPs from Osteo Sphere conditioned tissue culture medium will use heparin affinity chromatography as the initial basis for extraction and concentration of the OsteoSphere-produced BMPs.

What is claimed is:

1. A method for producing mineralized three-dimensional bone constructs, the method comprising:
(a) introducing osteoblasts into a cylindrical culture vessel that rotates about a central horizontal axis, said cylindrical culture vessel comprising a matrix-free culture medium;
(b) culturing said osteoblasts in said cylindrical culture vessel during horizontal rotation at a rate effective to create low shear conditions and to promote the formation of osteoblast aggregates comprising said osteoblasts;
(c) further culturing said osteoblast aggregates in said cylindrical culture vessel during horizontal rotation at a rate effective to create low shear conditions, whereby said osteoblast aggregates grow in size;
(d) introducing a matrix-free mineralization culture medium into said cylindrical culture vessel and culturing said osteoblast aggregates during horizontal rotation at a rate effective to create low shear conditions, wherein said mineralized three-dimensional bone constructs are formed.

2. A mineralized three-dimensional bone construct comprising a spheroid of between about 100 μm and about 4 mm in diameter comprising an inner core surrounded by an outer layer, wherein said inner core comprises osteoblasts, osteocytes, or both osteoblasts and osteocytes embedded within a crystalline matrix, wherein said crystalline matrix comprises calcium, phosphates, and carbonates, and wherein said outer layer comprises osteoblasts and does not include osteoclasts.
